SEQUENCE LISTING (9 sequences)

SEQ ID NO: 1 - DNA, 1539 bp, Influenza A virus
atggaccaaatttgcattggttaccatgcaaacaactcaacagagcaggttgacacaataatggaaaagaatgttactgtcacgcatgcccaagacatactggaaaagacacacaatgggaagctctgcagtctaaatggagttaagcctctcattttgagggattgtagtgtagctggatggctcctcggaaaccccatgtgtgatgaattcctcaatgtgccggaatggtcttacatagtggagaaggacagcccaatcaatggcctctgctacccaggggatttcaacgactatgaagagctgaaacacctgttgagcagtacgaaccattttgagaaaattcaaatcatccccaggagttcttggtccaatcatgatgcctcatcaggagtgagctccgcatgtccatataatgggaggtcctcctttttcagaaatgtagtgtggctcatcaaaaagaacaatgcatacccaacaataaaaaggaattacaataatactaaccaagaagatcttttggtactgtgggggattcaccatcctaatgatgcaacagagcagacaaagctctatcaaaacccaaccacctatgtttctgttggaacatcaacactgaaccagagatcggtcccagaaatagctaccaggcccaaagtaaatgggcaaagtggaagaatagagtttttctggacaatcttaaagccaaatgatgccatcaatttcgagagtaatggaaattttattgctccagaatatgcatacaaaattgccaagaaaggagactcagcaatcatgaaaagtggattggagtatggtaaccgcaacaccaagtgtcaaactccaatgggtgcaataaactccagcatgccatttcacaacatacaccctctcaccattggggaatgccccagatacgtgaagtcagatagattagtccttgcaacagggctcaggaatgtccctcaaagagaaacaagaggactatttggggccatagcaggcttcatagaaggagggtggcaaggaatggtagacggttggtatggataccaccatagcaacgagcaagggagtggatacgctgcagacaaagagtccactcaaaaggcaatagatggaatcactaataaggtcaactcaatcattgacaaaatgaacactcagtttgaggccgttggaaaggaatttaataacttagaaaggaggatagagaatttgaacaagaaaatggaagacggattcctagatgtctggacttataatgctgaacttctggttctcatggaaagtgagagaaccctagactttcatgactcaaatgtcaagaacctttacgacaaggttcgactacagcttagggataatgcaaaggagctgggtaatggttgtttcgagttctatcacaaatgtgatgatgaatgtatggaaagtgtaagaaacggaacgtatgactacccgcagtattcagaagaggcaagactaaacagagaggaaataagtggagtaaaattggaatcaataggaacttaccaaata

SEQ ID NO: 2 - protein, 513 aa, Influenza A virus
MDQICIGYHANNSTEQVDTIMEKNVTVTHAQDILEKTHNGKLCSLNGVKPLILRDCSVAGWLLGNPMCDEFLNVPEWSYIVEKDSPINGLCYPGDFNDYEELKHLLSSTNHFEKIQIIPRSSWSNHDASSGVSSACPYNGRSSFFRNVVWLIKKNNAYPTIKRNYNNTNQEDLLVLWGIHHPNDATEQTKLYQNPTTYVSVGTSTLNQRSVPEIATRPKVNGQSGRIEFFWTILKPNDAINFESNGNFIAPEYAYKIAKKGDSAIMKSGLEYGNRNTKCQTPMGAINSSMPFHNIHPLTIGECPRYVKSDRLVLATGLRNVPQRETRGLFGAIAGFIEGGWQGMVDGWYGYHHSNEQGSGYAADKESTQKAIDGITNKVNSIIDKMNTQFEAVGKEFNNLERRIENLNKKMEDGFLDVWTYNAELLVLMESERTLDFHDSNVKNLYDKVRLQLRDNAKELGNGCFEFYHKCDDECMESVRNGTYDYPQYSEEARLNREEISGVKLESIGTYQI

SEQ ID NO: 3 - DNA, 1482 bp, Influenza A virus
gcgcttcaaggcactaaacgatcttatgaacagatggaaactggtggagaacgccagaatgccactgagatcagagcatctgttggaagaatggttggtggaattgggaggttttacatacagatgtgcactgagctcaaactcagcgactatgaaggaaggctgattcagaacagcataacaatagagagaatggtcctctctgcatttgatgaaaggaggaacaaatacctggaagaacatcccagtgcggggaaggacccgaagaaaactggaggtccaatttatcgaaggagagacgggaaatggatgagagagctgattctatatgacaaagaggaaatcaggaggatctggcgtcaagcgaacaatggagaagacgcgactgctggtctcactcacctgatgatctggcattccaatctaaatgatgccacataccagagaacaagagctcttgtgcgtactggaatggaccccagaatgtgctctctgatgcaaggatcaactctcccgaggagatctggagctgctggtgcagcagtaaagggagtcggaacgatggtgatggaactaattcggatgataaaacgagggatcaatgatcggaatttctggagaggcgaaaatggacgaagaacaaggattgcatatgagagaatgtgcaacatcctcaaagggaaattccaaacagcagcacaaagagcaatgatggaccaggtgcgagaaagcagaaatcctgggaatgctgaaatcgaagatctcatcttcctggcacggtctgcactcatcctgagaggatcagtggcccataagtcctgcttgcctgcgtgtgtgtacggacttgctgtggccagtggatatgactttgagagagaagggtactctctagtcggaatagaccctttccgtctgcttcaaaacagccaggtcttcagtctcattagaccaaatgagaatccagcacacaagagtcaattggtgtggatggcatgccattctgcagcatttgaagacctgagagtctcaagtttcatcagagggacaagagtagtcccaagaggacaactatccaccagaggagttcaaattgcttcaaatgagaacatggaaacaatggactccagcactcttgaactgaggagcagatattgggctataaggaccaggagtggaggaaacaccaatcaacagagagcatctgcaggacaaatcagtgtacagcctactttctcggtacagagaaatcttcccttcgaaagagcgaccattatggcggcgttcacagggaatactgagggcagaacatctgacatgaggactgaaatcataaggatgatggaaagtgccagaccagaggatgtgtctttccaggggcggggagtcttcgagctctcggacgaaaaggcaacgaacccgatcgtgccttcctttgacatgagtaatgaaggatcttatttcttcggagacaatgcagaggag

SEQ ID NO: 4 - protein, 494 aa, Influenza A virus
ALQGTKRSYEQMETGGERQNATEIRASVGRMVGGIGRFYIQMCTELKLSDYEGRLIQNSITIERMVLSAFDERRNKYLEEHPSAGKDPKKTGGPIYRRRDGKWMRELILYDKEEIRRIWRQANNGEDATAGLTHLMIWHSNLNDATYQRTRALVRTGMDPRMCSLMQGSTLPRRSGAAGAAVKGVGTMVMELIRMIKRGINDRNFWRGENGRRTRIAYERMCNILKGKFQTAAQRAMMDQVRESRNPGNAEIEDLIFLARSALILRGSVAHKSCLPACVYGLAVASGYDFEREGYSLVGIDPFRLLQNSQVFSLIRPNENPAHKSQLVWMACHSAAFEDLRVSSFIRGTRVVPRGQLSTRGVQIASNENMETMDSSTLELRSRYWAIRTRSGGNTNQQRASAGQISVQPTFSVQRNLPFERATIMAAFTGNTEGRTSDMRTEIIRMMESARPEDVSFQGRGVFELSDEKATNPIVPSFDMSNEGSYFFGDNAEE

SEQ ID NO: 5 - DNA, 31 bp, Artificial
  An artificially synthesized primer sequence
gccctacgtagaccaaatttgcattggttac

SEQ ID NO: 6 - DNA, 34 bp, Artificial
  An artificially synthesized primer sequence
atagtttagcggccgcttatatttggtaagttcc

SEQ ID NO: 7 - DNA, 33 bp, Artificial
  An artificially synthesized primer sequence
acgcgtcgacgcgcttcaaggcaccaaacgatc

SEQ ID NO: 8 - DNA, 30 bp, Artificial
  An artificially synthesized primer sequence
cccaagcttctcctctgcattgtctccgaa

SEQ ID NO: 9 - DNA, 18 bp, Artificial
  An artificially synthesized nucleotide sequence
gtcgttgtcgttgtcgtt

(19) United States
(12) Patent Application Publication
(10) Pub. No.: US 2011/0195091 A1
(43) Pub. Date: Aug. 11, 2011

(54) Avian Influenza Virus Antigen, and Booster Immunization Method for Avian Influenza Vaccine in Combination with Mucosal Adjuvant Which is Effective Through Oral Administration

(75) Inventors: Yukihiro Miyoshi, Saitama (JP); Akira Ito, Saitama (JP); Kentaro Susa, Saitama (JP); Naomi Himeno, Saitama (JP); Toru Gotanda, Saitama (JP)

(73) Assignee: THE KITASATO INSTITUTE, Tokyo (JP)

(21) Appl. No.: 13/059,777

(22) PCT Filed: Aug. 13, 2009
(86) PCT No.: PCT/JP2009/064285

§ 371 Date: Apr. 27, 2011

(30) Foreign Application Priority Data
Aug. 18, 2008 (JP) 2008-210021

(51) Int. Cl. A61K 39/145; A61P 37/04

(52) U.S. Cl. 424/209.1

(57) ABSTRACT

When ELISA antibody titer dropped in chickens intramuscularly inoculated with an oil vaccine, recombinant HA and NP antigens prepared using gene recombinant technology were orally administered in combination with synthetic oligo CpG as a mucosal adjuvant to the chickens. Even when the immune response induced with the oil vaccine was impaired, the antibody titer was re-induced by orally administering the recombinant HA and NP antigens, and synthetic CpG-ODN in combination.

### TECHNICAL FIELD

The present invention relates to vaccination to poultry, to chickens in particular. Specifically, the present invention relates to immunization materials and methods for booster immunization targeting poultry subjected to an initial immunization with an injectable oil vaccine.

### BACKGROUND ART

By 2008, influenza infecting birds was spreading in and around Southeast Asia, hi particular, poultry farms sustained devastating damages (Bagchi, (2008) CMAJ, 178:1415). Furthermore, there were some human infections by the highly pathogenic subtypes of H5N1. A new influenza pandemic was feared (Wright, (2008) N. Engl. J. Med., 358:2540-2543), Under these circumstances, countermeasures against avian influenza virus became an important public health objective.

Induction of immunity by vaccination is an effective preventive measure for avian influenza. Hence, Japan has also produced and has a stockpile of an intramuscular injectable oil vaccine. If the immunity induced by this vaccination can be maintained at high levels over a long period, it will serve as a very effective preventive measure. However, conventional methods are insufficient in terms of effectiveness, cost, convenience, etc.

Methods for preparing materials for influenza vaccine include, for example, those described in “VACCINE USING SENDAI VIRUS VECTOR, AND VACCINE PROTEIN (Japanese Patent Application Kokai Publication No. (JP-A) 2000-253876 (unexamined, published Japanese patent application))”, “RECOMBINANT INFLUENZA VIRUSES FOR VACCINES AND GENE THERAPY (JP-A (Kokai) 2003-528570)”, and “METHOD FOR DEVELOPING PROTEIN VACCINES AND VACCINES FOR AVIAN INFLUENZA USING REVERSE GENETIC METHOD (JP-A (Kokai) 2007-282636)”. However, none of the documents mentions booster immunization.

Meanwhile, A. D. Altstein et al. undertook an attempt at booster immunization with recombinant NP in mice, but the administration route was intraperitoneal administration (Altstein et al. (2006) Arch. Virol. 151: 921-931). G. Le Gall-Recule et al also performed booster immunization with oil vaccine in chickens, but this administration route was subcutaneous inoculation (G. Le Gall-Recule et al. (2007) Avian Dis. 51:490-494). Y. Asahi-Ozaki et al. prepared an HA vaccine using recombinant virus produced by reverse genetics, and they reported a result obtained by testing the vaccine with nasal administration (Y. Asahi-Ozaki et al. (2006) Microbes Meet. 8:2706-2714).

As described above, there is no report of an effective case featuring an oral booster immunization method using recombinant protein antigens targeting chickens.

Prior art documents related to the present invention include:

### PRIOR ART DOCUMENTS

### Patent Documents

Patent Document 1: JT-A (Kokai) 2000-253876
Patent Document 2: JP-A (Kokai) 2003-528570
Patent Document 3: JP-A (Kokai) 2007-282636

### Non-Patent Documents

Non-patent Document 1: Bagchi, (2008) CMAJ, 178:1415
Non-patent Document 2: Wright, (2008) N. Engl. J. Med., 358:2540-2543
Non-patent Document 3: Altstein et al. (2006) Arch. Virol. 151: 921-931
Non-patent Document 4: G. Le Gall-Recule et al. (2007) Avian Dis. 51:490-494
Non-patent Document 5: Y. Asahi-Ozaki et al. (2006) Microbes Infect. 8:2706-2714
Non-patent Document 6: Eliasson D. G. et al. (2008) Vaccine 26:1243-52
Non-patent Document 7: Sylte M. J. et al. (2007) Vaccine 25:3763-72
Non-patent Document 8: OhbaK. et al. (2007) Vaccine 25:4291-300
Non-patent Document 9: Roy S. et al. (2007) Vaccine 25:6845-51
Non-patent Document 10: Huleatt J. W. et al. (2008) Vaccine 26:201-14
Non-patent Document 11: Webster R. G. et al. (1991) Vaccine 9:303-8
Non-patent Document 12: Ilyinskii R. O. et al. (2008) Vaccine 26:2177-85
Non-patent Document 13: Zanvit P. et al. (2008) Immunol. Lett. 115:144-52
Non-patent Document 14: Isaka M. et al. (2008) Microbiol. Immunol 52:55-63
Non-patent Document 15: Horthongkham N. et al. (2007) J. Immune. Based Ther. Vaccines 5:10

### SUMMARY OF THE INVENTION

### Problems to be Solved by the Invention

An objective of the present invention is to provide immunization materials that enable longer maintenance of immunity induced by an initial immunization with an oil vaccine, at a high level, and to establish a convenient and efficient method for booster immunization.

### Means for Solving the Problems

Using recombinant DNA technology, the present inventors prepared HA and NP proteins, which are said to be antigens effective in preventing avian influenza. The mucosal adjuvant synthetic oligo CpG was combined with the recombinant HA and NP antigens and the mixture was orally administered to chickens that have been intramuscularly inoculated with an oil vaccine. The result showed that, even when the immune response induced by oil vaccine was impaired, antibody production was re-induced by oral administration of recombinant HA and NP antigens in combination with synthetic oligo CpG The vaccine of the present invention was effective when used for booster immunization, while it was poorly effective or ineffective when used for initial immunization.

Specifically, the present invention provides:
[1] an immunization material comprising an avian influenza virus antigen and a mucosal adjuvant effective for oral administration;
   [2] the immunization material of [1], wherein the avian influenza virus antigens are recombinant HA antigen and recombinant NP antigen;
   [3] the immunization material of [1] or [2] for preventing avian influenza;
   [4] the immunization material of any one of [1] to [3], which is used by oral administration;
   [5] the immunization material of any one of [1] to [4], which is used for booster immunization;
   [6] a method for re-inducing immunity induced by an initial immunization, wherein an immunization material of any one of [1] to [5] is additionally administered to an animal administered with an avian influenza vaccine;
   [7] the method of [6], wherein the animal is a bird;
   [8] the method of [6] or [7], wherein the booster administration is achieved by oral administration;
   [9] use of the immunization material of [1] to [5] in manufacturing an agent for re-inducing immunity induced by initial immunization in an animal administered with vaccine against avian influenza; and
   [10] use of the immunization material of [1] to [5] in a method for re-inducing immunity induced by initial immunization in an animal administered with vaccine against avian influenza.

### BRIEF DESCRIPTION OF THE DRAWINGS

FIG. 1 shows photographs depicting antigen purity and reactivity to commercial antibodies confirmed by SDS-PAGE and Western blotting after purifying HA antigen (1-A) expressed by recombinant yeast containing the HA of A/duck/Mongolia/54/01 (H5N2) strain as a gene donor and NP antigen (1-B) expressed by recombinant E. coli containing the NP of A/duck/Hokkaido/9/99 (H9N2) strain as a gene donor.

FIG. 2 shows graphs depicting quantified and plotted time courses of titers of anti-HA and anti-NP antibodies before and after oral administration of recombinant HA and NP antigens, and mucosal adjuvant to chickens inoculated with an oil vaccine against avian influenza.

### MODE FOR CARRYING OUT THE INVENTION

The present invention provides immunization materials containing avian influenza virus antigens and a mucosal adjuvant that is effective for oral administration. The immunization materials of the present invention are preferably used to prevent influenza.

Influenza is a life-threatening serious infection in patients with pre-existing diseases or aged persons. Furthermore, there have recently been epidemics of avian influenza, which is infection in birds. Among type A influenza virus infections, highly pathogenic avian influenza exhibits markedly strong pathogenicity in poultry such as chicken, quail, and turkey. The highly pathogenic avian influenza is defined as an avian influenza virus that kills chickens at a high probability when inoculated into the vein, or as a so-called highly-virulent virus having consecutive basic amino acids at the cleavage site in HA.

Type A influenza viruses are classified based on the type of HA (and type of NA). Highly pathogenic avian influenza viruses identified to date are limited to subtypes H5 and H7. These subtype viruses can mutate into highly-virulent forms after repeated cycles of infection. Thus, precaution should be taken against these viruses regardless of whether they are in the highly virulent form or less virulent form.

Influenza viruses that are originally non-infectious in humans, for example, subtype viruses H5 and H7 can mutate to become infectious and capable of propagating in humans. Such mutant viruses that have finally acquired the ability to cause infection at a high frequency from humans to humans are called new influenza viruses. Since humans have no immunity against such new influenza viruses, the infection can spread and may cause a world-wide epidemic (pandemic), with prospects of causing tremendous damage.

“Influenza virus antigen” refers to a recombinant antigen originating from influenza virus as gene donor, which has an ability to prevent the infection and onset of influenza. Influenza virus antigens that are already known to have the effect of preventing influenza include, for example, hemagglutinin (HA) antigen, neuramimidase (NA) antigen, NP (nuclear protein) antigen, and M protein (matrix protein) antigen.

An influenza virus antigen to be contained in an immunization material of the present invention may be any subtype virus as long as it allows oral booster immunization to re-induce the immunity or to produce the effect of resisting immunity impairment. Antigens applicable to the present invention include, but are not limited to, for example, (1) HA and NA, which are viral surface antigens, and (2) NP and M, which are antigens located in the internal structure of a virus. PA, PB1, PB2, and NS2 can also be used in the present invention. They may be used alone or in any combination of two or more.

Preferred influenza virus antigens to be contained in an immunization material of the present invention are HA antigen and NP antigen. The influenza virus antigens of the present invention are not limited to the above-described antigens. Spontaneous or artificially produced mutant antigens having an altered gene sequence or amino acid sequence are also included in the present invention, as long as they are antigens that are functionally equivalent to the HA or NP antigen. Herein, an antigen functionally equivalent to HA antigen refers to a protein having the same antigenicity as HA antigen. As used herein, an antigen functionally equivalent to NP antigen refers to a protein having the same antigenicity as NP antigen.

An influenza virus antigen to be contained in an immunization material of the present invention is preferably a recombinant HA antigen. Such recombinant HA antigens include, for example:

(a) a protein comprising the amino acid sequence of SEQ ID NO: 2;
(b) a protein encoded by a nucleic acid comprising the coding region having the nucleotide sequence of SEQ ID NO: 1;
(c) a protein comprising an amino acid sequence with a substitution, deletion, insertion, and/or addition of one or more amino acids in the amino acid sequence of SEQ ID NO: 2, which is functionally equivalent to a protein comprising the amino acid sequence of SEQ ID NO: 2; and
(d) a protein encoded by a nucleic acid that hybridizes under stringent conditions to a nucleic acid comprising the nucleotide sequence of SEQ ID NO: 1, which is functionally equivalent to a protein comprising the amino acid sequence of SEQ ID NO: 2.

An influenza virus antigen to be comprised in an immunization material of the present invention is preferably a recombinant NP antigen. Such recombinant NP antigens include, for example:

(a) a protein comprising the amino acid sequence of SEQ ID NO: 4;
(b) a protein encoded by a nucleic acid comprising the coding region having the nucleotide sequence of SEQ ID NO: 3;
(c) a protein comprising an amino acid sequence with a substitution, deletion, insertion, and/or addition of one or more amino acids in the amino acid sequence of SEQ ID NO: 4, which is functionally equivalent to a protein comprising the amino acid sequence of SEQ ID NO: 4; and
(d) a protein encoded by a nucleic acid that hybridizes under stringent conditions to a nucleic acid comprising the nucleotide sequence of SEQ ID NO: 3, which is functionally equivalent to a protein comprising the amino acid sequence of SEQ ID NO: 4.

Known methods which cause random mutations to a given nucleotide sequence include substitutions) of base pair(s) through nitrous acid treatment of DNA (Hirose, S. et al., Proc. Natl. Acad. Sci. USA., 79:7258-7260, 1982). This method enables random introduction of substitutions) of base pair(s) into a specific segment by nitrous acid treatment of the segment desired to be mutated. Alternatively, technologies for site-directing a target mutation include the gapped duplex method (Kramer W. and Fritz H J., Methods in Enzymol., 154:350-367, 1987) and the like. A cyclic double stranded vector in which a gene to be introduced with a mutation is cloned, is separated into single strands. These single strands are hybridized with a synthetic oligonucleotide mutated at the target site. A vector-derived complementary single strand DNA cleaved and linearized by a restriction enzyme is annealed with the cyclic single stranded vector, and the gap between the oligonucleotide and the vector is filled by using a DNA polymerase, which is then made into a complete double stranded vector by ligation.

The number of amino acids to be modified would be typically within 50 amino acids, preferably within 30 amino acids, and more preferably within 5 amino acids (for example, 1 amino acid).

When an amino acid is artificially substituted, substitution with an amino acid having similar properties would result in maintaining the activity of the original protein. Proteins of the present invention include proteins resulting from a conservative substitution in the above substitution of amino acid(s), and which are functionally equivalent to the proteins comprising the amino acid sequence of SEQ ID NO: 2 or 4, Conservative substitution is considered important when substituting amino acid(s) of domains important for protein activities, etc. Such a conservative substitution of amino acid(s) is well known to those skilled in the art.

Examples of amino acid groups suitable for conservative substitution include basic amino acids (such as lysine, arginine, and histidine), acidic amino acids (such as aspartic acid and glutamic acid), uncharged polar amino acids (such as glycine, asparagine, glutamine, serine, threonine, tyrosine, and cysteine), nonpolar amino acids (such as alanine, valine, leucine, isoleucine, proline, phenylalanine, methionine, and tryptophane), β branched amino acids (such as threonine, valine, and isoleucine), and aromatic amino acids (such as tyrosine, phenylalanine, tryptophane, and histidine).

Moreover, non-conservative substitution may increase protein activities or such (for example, constitutively activated proteins).

In addition, proteins which are functionally equivalent to the proteins comprising the amino acid sequence of SEQ ID NO: 2 or 4 can be obtained by methods that utilize hybridization. That is to say, a DNA encoding an influenza virus antigen of the present invention as shown in the SEQ ID NO: 1 or 3, or a fragment thereof is used as a probe, and then DNAs that can hybridize to them are isolated. A hybridization reaction performed under stringent conditions leads to the selection of highly homologous DNA as a nucleotide sequence. This increases the chances of isolated proteins containing proteins that are functionally equivalent to the influenza virus antigen. Examples of a highly homologous nucleotide sequence include those having 70% or more, and desirably 90% or more homology (identity).

In a specific example, the term “stringent conditions” refers to hybridization conditions with 6×SSC, 40% formamide at 25° C and subsequent washing with 1×SSC at 55° C. The stringency depends on conditions such as salt concentration, formamide concentration, or temperature; however it is apparent for those skilled in the art to set these conditions so as to obtain a necessary stringency.

With the use of hybridization, for example, DNAs encoding homologues of the influenza virus antigens other than those proteins comprising the amino acid sequence of SEQ ID NO: 2 or 4 can be isolated.

Proteins which are functionally equivalent to a protein comprising the amino acid sequence of SEQ ID NO: 2 or 4 normally have a high homology with the amino acid sequence of SEQ ID NO: 2 or 4. The term “high homology” refers to a sequence homology (identity) of at least 30% or more, preferably 50% or more, more preferably 80% or more (for example, 95% or more). The homology of the nucleotide sequences and amino acid sequences can be determined using a homology search site via the internet (For example, homology searches such as FASTA, BLAST, PSI-BLAST, and SSEARCH can be used in the DNA Data Bank of Japan (DDBJ) [examples of which include the homology search page (Search and Analysis) at the DNA Data Bank of Japan (DDBJ) website; http://www.ddbj.nig.acjp/E-mail/homology-j.html]. Furthermore, searches using BLAST can be carried out through the web site of the National Center for Biotechnology Information (NCBI) [examples of which include BLAST page at the homepage of NCBI website; http://wvw.ncbi.nlm.nih.gov/BLAST/; Altschul, S. F. et al., J. Mol. Biol., 1990, 215 (3):403-10; Altschul, S. F. & Gish, W., Meth. Enzymol., 1996, 266:460-480; Altschul, S. F. et al, Nucleic Acids Res., 1997, 25:3389-3402]).

For example, in the calculation of the identity of amino acid sequences using Advanced BLAST 2.1, the identity value (%) can be obtained by the following: blastp is used as the program, expect value is set at 10, all filters are set at OFF, BLOSUM62 is used for matrix, and gap existence cost, per residue gap cost, and lambda ratio are set at 11, 1, and 0.85, respectively (default parameters) (Karlin, S. and S. F. Altschul (1990) Proc. Natl. Acad. Sci. USA 87:2264-68; Karlin, S. and S. F. Altschul (1993) Proc. Natl. Acad. Sci. USA 90:5873-7).

The recombinant antigens of the present invention are not particularly limited, as long as they are suitable for oral administration. The antigens include, for example, the antigens expressed by recombinant DNA technologies in unicellular prokaryotes such as E. coli or unicellular eukaryotes such as yeast, and the antigens expressed in multicellular organisms such as insect, plant, and animal cells.

When eukaryotic cells are used, host cells include, for example, animal cells, plant cells, and fungal cells. Known animal cells include mammalian cells such as CHO (J. Exp. Med. 108, 945 (1995)), COS, 3T3, myeloma, BHK (baby hamster kidney), HeLa, and Vero; amphibian cells such as Xenopus laevis oocytes (Valle, et al., Nature 291, 358-340 (1981)); and insect cells such as Sf9, Sf21, and Tn5. In the present invention, CHO-DG44, CHO-DXB11, COS7, and BHK cells are preferably used. Of the animal cells, CHO cells are particularly preferable for large-scale expression. Vectors can be introduced into host cells, for example, by calcium phosphate methods, DEAE-dextran methods, methods using cationic liposome DOTAP (Boehrmger-Mannheim), electroporation methods, lipofection methods, etc.

It is known that plant cells such as Nicotiana tabacum-derived cells are protein production systems, and callus cultures from these cells may be used. Protein systems using fungal cells including yeasts, for example, the genus Saccharomyces such as Saccharomyces cerevisiae and Saccharomyces pombe; and filamentous fungi, for example, the genus Aspergillus such as Aspergillus niger are known.

When prokaryotic cells are used, production systems that use bacterial cells are available. Such bacterial cells include E. coli, for example, JM109, DH5α, and HB101, and Bacillus subtilis. An antigen of the present invention can be prepared by culturing cells transformed with a DNA of the present invention in vitro and purifying the resulting antigen by methods normally used by those skilled in the art.

“Mucosal adjuvant” refers to a substance that enhances the immune response when administered to mucosa. An immune adjuvant may also be referred to as an adjuvant, adjuvant for vaccination, vaccination adjuvant, vaccine adjuvant, immune adjuvant composition, adjuvant composition, composition for adjuvant, immunostimulant, or immunopotentiator. All of these substances refer to the immune adjuvant of the present invention. Mucosal adjuvants included in the immunization materials of the present invention include, but are not limited to, for example, synthetic oligo CpG (Ameiss et al., (2006) Vet. Immunol. Immunopathol. 110:257-67), lectin, liposomes, E. coli heat labile toxin (or cholera toxin), and interleukin-12. A preferred mucosal adjuvant to be contained in an immunization material of the present invention is synthetic oligo CpG

The present invention provides methods for re-inducing the immunity induced by initial immunization in animals administered with vaccine against avian influenza, by additionally administering an immunization material containing avian influenza virus antigens and a mucosal adjuvant effective in oral administration. Thus, the immunization material of the present invention is used for booster immunization.

Immunization materials of the present invention exert their effects when administered (booster immunization) to individuals in which the antibody titer has been already elevated by vaccination (for example, administration by injection). The administered immunization materials can further elevate the antibody titer or maintain it at high levels in the individuals. For example, even when a high-level antibody titer already begins to drop in individuals inoculated with oil vaccine in initial immunization, the antibody titer can be significantly re-elevated in the individuals by orally administrating recombinant antigens and a mucosal adjuvant.

The vaccine efficacy can be assessed by antibody titer assay using ELISA, HI titer assay, neutralization antibody titer assay, or clinical observation or survival assessment in viral attack test using mice or the like. These methods are known to those skilled in the art.

Subject animals to be administered with immunization materials of the present invention include, for example, humans, monkeys, birds, horses, and pigs. Preferred subjects to be administered with immunization materials of the present invention are birds.

The administration route for the immunization materials of the present invention is mucosal administration, and a preferred route is oral administration.

Immunization materials of the present invention can be formulated according to the usual methods (for example, Remington's Pharmaceutical Science, latest edition, Mark Publishing Company, Eastern, U.S.A), and may contain pharmaceutically acceptable carriers and additives together. Examples include surfactants, excipients, coloring agents, preservatives, stabilizers, buffers, suspending agents, and isotonizing agents, although they are not limited thereto and other common carriers may be appropriately used.

A recombinant antigen prepared using as a gene donor an influenza virus of the present invention may be administered orally after formulating it in combination with other ingredients. Such ingredients used in combination include, for example, feed for the target animal, flavor-improving additives (lactose, etc.), and drinking water.

The dose varies depending on the type of animal which receives administration. For example, when a recombinant antigen derived from influenza virus is used in crude or partially purified form, chickens are conveniently fed a diet prepared by mixing ordinary feed with the antigen at a weight ratio of around 5% (“ORAL VACCINE AGAINST PROTOZOIASIS USING GENETICALLY MODIFIED PLANT (International Application No. WO 2005/116216)”). The single dose can be selected in the range of 1 to 100 mcg (microgram; the same abbreviation is used hereinafter) of purified protein per bird, when the antigen is administered, for example, three to 14 times for three to 14 days. However, the dose is not limited to such doses. The antigen may be administered consecutively or at any interval.

For other animals, the antigen can be administered at a dose determined based on the body weight or body surface area. However, the dose is not limited to such doses. The appropriate dose can be determined by considering the expression level of the protective antigen, optimal antibody production, properties of formulated feed, and the like.

Use of immunization materials of the present invention may be described as follows:

(1) use of an immunization material comprising avian influenza virus antigens and a mucosal adjuvant effective in oral administration in manufacturing agents for re-inducing the immunity induced by initial immunization in animals administered with vaccine against avian influenza; or
(2) use of an immunization material comprising avian influenza virus antigens and a mucosal adjuvant effective in oral administration in methods for re-inducing the immunity induced by initial immunization in animals administered with vaccine against avian influenza.

All prior art documents cited in the specification are incorporated herein by reference.

### EXAMPLES

The present invention is specifically illustrated below with reference to Examples, but it is not to be construed as being limited thereto.

### Example 1

### Preparation of Mucosal Adjuvant and Recombinant Antigen Proteins Originating from Influenza Virus

### (1) Preparation of Recombinant HA Antigen

The gene encoding the amino acids of positions 17 to 528 relative to the N terminus in the HA protein of A/duck/Mongolia/54/01 (H5N2) strain was amplified by reverse transcription-PCR.

Specifically, reverse transcription-PCR was carried out using One Step RNA PGR kit (AMV) (TaKaRa) with primers 5′-GCC CTA CGT AGA CCA AAT TTG CAT TGG TTA C-3′ (SEQ ID NO: 5) and 5′-ATA GTT TAG CGG CCG CTT A TAT TTG GTA AGT TCC-3′ (SEQ ID NO: 6),

The resulting cDNA fragment (nucleotide sequence/SEQ ID NO: 1; amino acid sequence/SEQ ID NO: 2) was digested with restriction enzymes SnaBI and NotI, and then inserted into plasmid pPIC9K (Multi-Copy Pichia Expression Kit; Invitrogen). The constructed expression plasmid for HA was introduced into Pichia pastoris GS115 strain (Multi-Copy Pichia Expression Kit; Invitrogen). This transformation experiment was conducted according to the instruction manual appended to the Multi-Copy Pichia Expression Kit

A clone expressing recombinant HA was selected from the obtained transformants by Western blotting using Rabbit Polyclonal anti-Avian Influenza A Hemagglutinin antibody (Novus) and Goat anti-Rabbit IgG-heavy and light chain cross-adsorbed Antibody HRP Conjugated (Bethyl Laboratories).

The obtained HA-expressing yeast was inoculated to YPD medium (2% glucose, 1% yeast extract, 2% peptone) and cultured at 28° C with shaking overnight. The culture medium was inoculated to MGY medium (1% glycerol, 0.00004% biotin, yeast nitrogen base (Invitrogen)), and the yeast was cultured at 28° C with shaking until ODgoo reached 2.0 from 1.5 (for 16 to 18 hours). The culture medium was aseptically centrifuged (2,000×g for five minutes) to harvest the microbial cells. The resulting yeast cells were re-suspended in BMMY medium (0.5% methanol, 1% yeast extract, 2% peptone, 0.00004% biotin, yeast nitrogen base (Invitrogen), 100 mM phosphate buffer (pH6.0)). This suspension was incubated at 28° C with shaking for 16 hours, and then methanol (0.5% of the culture medium volume) was aseptically added. After six hours of culture, the obtained yeast cells were harvested by centrifugation at 5,000×g for 15 minutes, and frozen at −80° C. The cells were thawed and re-suspended in breaking buffer (50 mM Tris-Cl, 1 mM EDTA, 5% glycerol (pH 8.0)). Glass beads (425 to 600 microns; Sigma) were added to the suspension. The cells were crushed in Bioruptor (Cosmo bio) by three rounds of 30 cycles of sonication for 30 seconds and resting for 30 seconds. The extract was centrifuged (14,000×g for 30 minutes), and the supernatant was collected and clarified using a filter with 0.22-mcm (micrometer, the same abbreviation is used hereinafter) pores. The resulting sample was used as crude extract.

The crude extract was fractionated in HiPrep 16/10 Q XL column (GE healthcare). The binding buffer used was 20 mM Tris-Cl (pH 8.0). Elution was achieved using an elution buffer of 20 mM Tris-Cl (pH 8.0) with an NaCl concentration gradient up to 1M. Fractions containing recombinant HA were selected by Western blotting using the same antibodies described above.

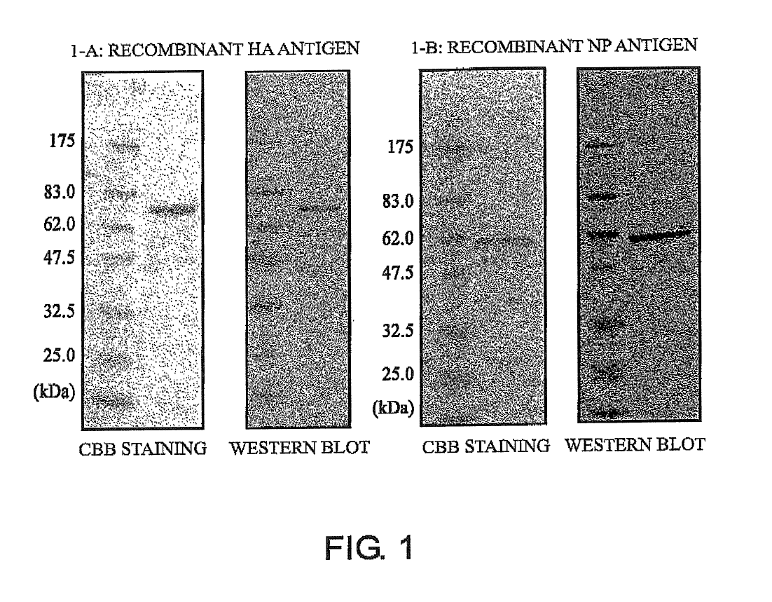

The collected fractions were concentrated and dialyzed by ultrafiltration (cut-off molecular weight, 30 kDa), The buffer was replaced with 20 mM Tris-Cl (pH 8.0) containing 150 mM NaCl. The purity of the prepared recombinant HA antigen was confirmed by 10% SDS-PAGE and Western blotting (FIG. 1 (1-A)).

### (2) Preparation of Recombinant NP Antigen

The NP gene was prepared from A/duck/Hokkaido/9/99 (H9N2) strain by reverse transcription-PCR using One Step RNA PCR kit (AMV) (TaKaRa) with 5′-ACG CGT CGA CGC GCT TCA AGG CAC CAA ACG ATC-3′ (SEQ ID NO: 7) and 5′-CCC AAG CTT CTC CTC TGC ATT GTC TCC GAA-3′ (SEQ ID NO: 8).

The cDNA fragment (nucleotide sequence/SEQ ID NO: 3; amino acid sequence/SEQ ID NO: 4) of the NP gene was digested with restriction enzymes SalI and HindIII, and inserted into plasmid vector pQE31 (Qiagen) for expressing a recombinant protein in E. coli. The recombinant DNA was introduced into Escherichia coli M15 strain to prepare recombinant E. coli expressing recombinant NP. This recombination experiment was conducted according to the instruction manual provided by Qiagen.

The obtained recombinant NP-expressing E. coli was inoculated in LB medium (1.0% tryptone, 0.5% yeast extract, 1.0% sodium chloride, 100 mcg/ml ampicillin sodium salt, 25 mcg/ml kanamycin sulfate), and cultured at 37° C with shaking overnight. This pre-culture was inoculated in a 100-times larger volume of fresh LB medium. The medium was incubated with shaking at 37° C. until OD₆₀₀ reached 0.6. To grow the bacteria until the turbidity reached a desired level, IPTG was added at a final concentration of 1 mM to the medium and the bacteria were incubated at 37° C with shaking overnight to induce the expression of NP protein. After culture, the bacterial cells were harvested by centrifugation at 5,000×g for 20 minutes. The bacterial cells were re-suspended in 20 mM Tris-HCl buffer (pH 8.0), and lysozyme, a bacteriolysis enzyme, was added thereto at a final concentration of 0.4 mg/ml. The suspension was incubated on ice for 30 minutes for bacteriolysis. After the reaction, sodium deoxychlorate was added at a final concentration of 0.1%. The mixture was incubated at 37° C. for 30 minutes. After treating the bacterial lysate with Polytron (8,000 rpm for two minutes), the lysate was centrifuged at 10,000×g for 30 minutes.

50 ml of the resulting extract was combined with 5 ml of Ni-NTA Agarose (Qiagen), The mixture was incubated at 4° C. for one hour for binding, and loaded onto a polypropylene column (5 ml). The column was washed with 20 ml of washing buffer A (20 mM Tris-HCl, 0.5 M NaCl, 20 mM imidazole (pH 8.0)) and washing buffer B (20 mM Tris-HCl, 0.5 M NaCl, 75 mM imidazole (pH 8.0)). Then, 2.5 ml of elution buffer (20 mM Tris-HCl, 0.5 M NaCl, 250 mM imidazole (pH 8.0)) was loaded four times onto the column to elute recombinant NP. The obtained antigen protein was used after dialyzing against 20 mM Tris-HCl (pH8.0) buffer containing 0.5 M NaCl. The purity of the prepared recombinant NP antigen was confirmed by 10% SDS-PAGE and Western blotting (FIG. 1 (1-B)).

### (3) Preparation of Mucosal Adjuvant

The CpG sequence 5′-GTC GTT GTC GTT GTC GTT-3′ (Ameiss et al., (2006) Vet. Immunol. Immunopathol. 110:257-67; SEQ ID NO: 9) which has been reported to produce the effect of enhancing the immunity when used as a mucosal adjuvant by oral administration was used hi the present invention. The DNA (CpG-oligo dinucleotide; hereinafter abbreviated as CpG-ODN) was designed to have sulfur atoms in the phosphate backbone instead of oxygen atoms to enhance the resistance to degradation. The synthesis of the DNA was ordered to Operon Co.

After ethanol precipitation, the synthesized CpG-ODN was dissolved in sterile PBS (137 mM NaCl, 8.1 mMNa₂HPO₄, 2.68 mM KCl, 1.47 mM KH₂PO₄), and stored at −20° C until use in experiments.

### Example 2

### Oral Administration of Recombinant Antigens and Mucosal Adjuvant to Chickens Inoculated with Oil Vaccine

An oil vaccine against avian influenza was inoculated to four-week-old white leghorns (line-M) as initial immunization. Specifically, 10⁴ TCID50 of virus was prepared using avian influenza virus A/duck/Hokkaido/Vac-1/04 (H5N1) strain as seed virus, and 0.2-ml aliquots of the virus were each inoculated into the allantoic cavities of 11-day-old embryonated hen eggs. The eggs were incubated at 34° C for 72 hours, and then allowed to stand at 4° C. overnight. The allantoic fluid was harvested from the hen eggs after infection, and centrifuged (3,000×g for 20 minutes) to obtain the supernatant. After adding β-propiolactone at a final concentration of 0.05% (v/v), to inactivate the virus, the supernatant was incubated at 4° C. for five days. The inactivated viral suspension was assayed for the HA titer, and then diluted to 5,120 HAU/ml with PBS. The resulting suspension was used as an antigen solution. Light liquid paraffin, tetraoleic acid polyoxyethylene (40) sorbitol, and sorbitan sesquioleate were added at concentrations of 65%, 1.4% (w/v), and 2.0% (w/v), respectively, to the antigen solution. The mixture was emulsified with an emulsifier device (circulation flow volume of 41/min, 8,000 rpm, 0.5 kPa) to give a vaccine. The HA titer of the oil vaccine was 1,024 HAU/ml. 0.5 ml of the vaccine was intramuscularly inoculated to each chicken. Specifically, the dose of antigen per bird was 512 HAU.

The immune response induced by the vaccination in the subject chickens was monitored by venous blood sampling at one-week intervals, followed by EUSA for the antibody titer. Specifically, recombinant HA or NP antigen was diluted to 0.1 mcg/ml with 0.05 M sodium carbonate buffer (1.59 g/l NaaHCOs, 2.93 g/l NaH₂CO₃ (pH 9.6)), and aliquoted in 96-well ELIS A plates (IWAKI). The plates were allowed to stand at 4° C. overnight for coating. After the plates were washed with PBS-T buffer (0.05% Tween20, 137 mM NaCl, 8.1 mM Na₂HPO₄; 2.68 mM KCl, 1.47 mM KH₂PO₄), a blocking solution prepared by adding bovine serum albumin at a final concentration of 3% (w/v) to PBS-T buffer was aliquoted into the plates. The plates were allowed to stand at 37° C for one hour for blocking. After blocking treatment, the plates were washed with PBS-T buffer. Then, bovine serum albumin was added at a final concentration of 0.3% (w/v) to PBS-T buffer to prepare an antibody dilution buffer. The sera of the subject chickens were diluted 100 fold up to 51,200 fold by serial twofold dilution and aliquoted into the plates. The plates were allowed to stand at 37° C for one hour for reaction with antigen. After the plates were washed with PBS-T buffer, HRPO-labeled anti-chicken IgG antibody (ZYMED) diluted 12,000 fold with the antibody dilution buffer was aliquoted into the plates. The plates were incubated at 37° C. for one hour. After the plates were washed with PBS-T buffer, a substrate solution (14.6 g/l Na₂HPO₄, 10.2 g/l citrate monohydrate, 1 g/l ortho-phenylenediamine, 1 ml/l hydrogen peroxide) was added thereto. The plates were allowed to stand at 37° C in the dark for 15 minutes. A termination solution (5N sulfuric acid) was added to stop the reaction. The absorbance at 492 nm was measured in a microplate reader (Corona MTP-120).

Recombinant HA and NP antigens and mucosal adjuvant CpG-ODN were orally administered to the chickens (during 19th week after immunization with oil vaccine) whose ELISA antibody titers dropped after the maximal level. Specifically, the following three groups were prepared: (i) a group administered with 100 mcg of recombinant HA, 100 mcg of recombinant NP, and 25 mcg of CpG-ODN; (ii) a group administered with 100 mcg of recombinant HA and 100 mcg of recombinant NP; and (iii) a group administered with PBS. Each group consisted of four chickens. Each antigen and CpG-ODN were adjusted to a total volume of 2 ml with PBS, and administered orally with a forced water load using a syringe. They were administered every day for consecutive seven days. The ELISA antibody titer was monitored over time during the administration period and one week after administration.

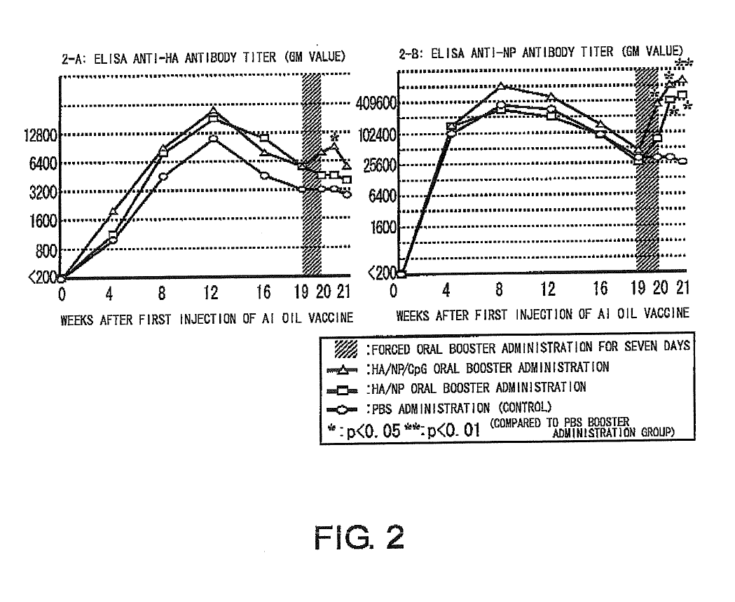

The experiment result is shown in FIG. 2.

This experiment result showed that, even when the immune response induced with oil vaccine was impaired, the antibody titer could be re-induced by orally administering the recombinant HA and NP, and CpG-ODN in combination (FIG. 2; p<0.05). Marked re-induction of immunity was observed particularly against NP, an antigen shared by all subtypes of type A influenza (FIG. 2 (2-B); p<0.01). The vaccine of the present invention was effective when used for booster immunization, while it was poorly effective or ineffective when used for initial immunization.

### INDUSTRIAL APPLICABILITY

The present invention provides immunization materials and booster immunization methods that enable longer maintenance of immunity induced by an initial immunization with an oil vaccine, at a high level. The immunization materials and booster immunization methods of the present invention enable convenient and efficient vaccination for preventing influenza.

1. An immunization material comprising an avian influenza virus antigen and a mucosal adjuvant effective for oral administration.

2. The immunization material of claim 1, wherein the avian influenza virus antigens are recombinant HA antigen and recombinant NP antigen.

3. The immunization material of claim 1 for preventing avian influenza.

4. The immunization material of claim 1, which is used by oral administration.

5. The immunization material of claim 1, which is used for booster immunization.

6. A method for re-inducing immunity induced by an initial immunization, wherein an immunization material of claim 1 is additionally administered to an animal administered with an avian influenza vaccine.

7. The method of claim 6, wherein the animal is a bird.

8. The method of claim 6, wherein the booster administration is achieved by oral administration.
